(19) United States
(12) Patent Application Publication
(10) Pub. No.: US 2021/0015178 A1
(43) Pub. Date: Jan. 21, 2021

(54) Performance Enhancing & Feet Protecting Athletic Sock

(71) Applicant: Jason Sunghyun WHANG, Helotes, TX (US)

(72) Inventor: Jason Sunghyun WHANG, Helotes, TX (US)

(21) Appl. No.: 16/839,073

(22) Filed: Apr. 2, 2020

Related U.S. Application Data
(60) Provisional application No. 62/827,946, filed on Apr. 2, 2019.

(51) Int. Cl. A41B 11/02; A41D 13/06; A61F 13/08; A41B 17/00

(52) U.S. Cl. CPC A41B 11/02; A41D 13/06; A63B 2071/1283; A41B 17/00; A61F 13/08

(57) ABSTRACT

An athletic sock for athletes, or those seeking to increase their athletic performance, to reduce the likelihood of lateral-extension ankle sprains and foot and leg cramps, and to reduce the likelihood of aggravating or bring relief from plantar fasciitis. The sock will additionally increase an athlete's vertical jump height and lateral foot speed. The sock enacts tensions on the foot at specific locations causing the foot to dorsiflex. The sock incorporates elastic material from the balls of the feet to an area ranging from the instep to above the ankle to provide tension for dorsiflexion. Elastic material is placed around the ankle and heel for support and protection while maintaining tensile strength that does not inhibit easy application of the sock or the foot's range of motion. Elastic material can be added around the arch of the sock to further reduce the aggravation of, or bring relief from, plantar fasciitis.

### CROSS REFERENCES TO RELATED APPLICATIONS

This application claims the benefit under Title 35 United States Code § 119(e) of U.S. Provisional Patent Application Ser. No. 62/827,946; Filed: Apr. 2, 2019; the full disclosure of which is incorporated herein by reference.

### BACKGROUND OF THE INVENTION

### 1. Field of the Invention

The present invention relates generally to socks that are worn by athletes when participating in competitive sports or recreational athletic activities but may also be relevant in other instances where socks are worn.

### 2. Description of the Related Art

There are no performance-enhancing athletic socks that increase an athlete's vertical jump height and lateral foot speed. One performance-enhancing athletic sock focuses on compression of the foot to increase blood flow and flow of oxygen to the foot resulting in reduced fatigue and pain. Other athletic socks on the market simply hope to increase athletic performance by wicking away sweat and by providing comfort and stability. Other forms of athletic performance enhancement are limited to shoes.

Currently, there is also a lack of an effective device that can reduce the likelihood of ankle injuries, and most forms of ankle protection, such as the ankle brace, aim to rehabilitate after the ankle has been injured. Furthermore, these ankle braces are often very stiff and uncomfortable to wear in competition. In fact, a number of studies have found that ankle braces decrease vertical jump height and broad jump distance, and slow the time of a sprint. Ankle braces have also been found to lead to knee problems because they constrict the range of motion of the foot thus preventing the foot from absorbing much of the shock.

There is also a lack of an effective device that can relieve or reduce the likelihood of the aggravation of plantar fasciitis or reduce the likelihood of foot and lower leg cramps. For plantar fasciitis, there are devices that act as a splint or a brace that extends up the leg to help alleviate the aggravation of plantar fasciitis, however, these implementations are large or unable to be used while continuously walking around. Though there is a sock that helps to prevent plantar fasciitis, it does so by implementing a heel support (see application publication KR 20140021949A) and does not use dorsiflexion. For foot and leg cramps, though there is a brace to prevent cramping of the foot or leg overnight (see application publication JP 2010234040A) and a few other methods that include a massaging apparatus or using oils, there are no socks that help to reduce the likelihood of foot and lower leg cramps.

Though none exists in the market, there are some prior efforts described in the literature for an ankle protection sock and a sock to prevent plantar fasciitis. These prior efforts are each unique in their implementations, but none of them increase the athlete's vertical jump height and lateral foot speed or reduce the likelihood of the combination of lateral-extension ankle sprains, foot and lower leg cramping, and the aggravation of plantar fasciitis.

### SUMMARY OF THE INVENTION

In fulfillment of the above objectives, the present invention provides an athletic sock intended for but not limited to competitive athletes, recreational athletes, or those either seeking to increase their athletic performance, to reduce the likelihood of lateral-extension ankle sprains and/or foot and/or leg cramps, and/or seeking to reduce the likelihood of aggravating or bring relief from plantar fasciitis. The sock will increase an athlete's vertical jump height and lateral foot speed, reduce the likelihood of lateral-extension ankle sprains, reduce the likelihood of aggravating or provide relief from plantar fasciitis, and reduce the likelihood of foot and/or leg cramps. The sock of the present invention accomplishes this by enacting tensions on the foot at specific locations causing the foot to dorsiflex. The sock incorporates elastic material from the balls of the feet to an area ranging from the instep of the foot to above the ankle to provide tension for dorsiflexion of the foot of up to approximately 45 degrees. Elastic material is also placed around the ankle and heel to model the formation of typical ankle brace for greater support and protection while maintaining at a tensile strength that does not inhibit easy application of the sock or the foot's range of motion. Finally, elastic material can be added optionally around the arch portion of the sock to further reduce the likelihood of aggravating or bring relief from plantar fasciitis.

In one embodiment of the present invention, the sock will increase an athlete's vertical jump height and lateral foot speed and reduce the likelihood of lateral-extension ankle sprains. In so doing, it will also reduce the likelihood of foot and/or lower leg cramps and provide relief from or reduce the likelihood of aggravating plantar fasciitis. The sock incorporates elastic material from the balls of the feet to an area ranging from the instep of the foot to above the ankle to provide tension for dorsiflexion of the foot of up to approximately 45 degrees. Elastic material is also placed around the ankle and heel to model the formation of a typical ankle brace for greater support and protection while maintaining a tensile strength that does not inhibit the foot's range of motion.

An athletic sock is a sock tailored for competitive sports or other athletic activities. Athletic socks are generally made from myriad fabrics to provide comfort and stability for the foot. They often incorporate strategically placed cushioning to provide impact protection and may be designed specifically for either the right or left foot. Though the fabrics that make up the sock tend to vary, they are usually designed to wick away perspiration from the foot and to prevent blisters. Most athletic socks are made from materials like polyester, nylon, wool, cotton, spandex, or a blend of the materials depending on the intended use of the sock as each fabric has different moisture-wicking properties and offer differing levels of comfort, ventilation and cushioning. The socks may also differ in height ranging from ankle socks, to quarter socks which are slightly higher, to crew socks and over-the-calf socks.

Further objectives of the present invention will become apparent from an understanding of the following detailed description and the attached drawing figures which may be briefly described as follows.

### BRIEF DESCRIPTION OF THE DRAWINGS

To assist those of skill in the art in making and using the disclosed invention and associated methods, reference is made to the accompanying figures, wherein:

FIG. 1 is a perspective schematic view of a first preferred embodiment of the performance enhancing, feet protecting sock of the present invention. The figure schematically shows the placement and direction of tension of the elastic material incorporated into the sock.

FIGS. 2A-2C are orthogonal views of the first embodiment of the sock of the present invention.

FIGS. 3A-3C are orthogonal views of a second embodiment of the sock of the present invention incorporating a plantar fasciitis option.

FIG. 4 is a perspective schematic view of the second preferred embodiment of the performance enhancing, feet protecting sock of the present invention. The figure schematically shows the placement and direction of tension of the elastic material incorporated into the sock, including tension around the arch for reducing the likelihood of aggravating and/or providing relief of plantar fasciitis.

The Drawing Figures show the use of elastic material fixed over or incorporated into the material of the sock with the following intended functions: elastic material causing dorsiflexion of all toes, including the pinky toe, beginning at the balls of the feet to an area above or near the instep; elastic material providing ankle support; elastic material providing further ankle stability; and elastic material providing additional support for plantar fasciitis

### DETAILED DESCRIPTION OF THE PREFERRED EMBODIMENTS

An increase in one's sprint speed and vertical jump can have a tremendous impact on athletes in almost every sport. For example, the difference between the times of the gold and silver medalists in the 100M men's sprint in the 2016 Olympics was decided by less than a tenth of a second and the difference in times between the silver and bronze medalists in the same event was decided by two hundredths of a second. That is a difference of 0.81%, 0.2% in their sprint times. In the same Olympics, the difference in the high jump heights for men was two hundredths of a meter or 0.8 inches. Any increase of 3-5% in sprint speed and 1-2 inches in jump height to these athletes' performance are enormous, however, this also extends to athletes from other sports and recreational athletes that strive to perform to the best of their ability each time they pull on their socks to compete.

According to a number of studies, ankle sprains are the most common sports injury with as many as 73% of all athletes in these studies having recurrent ankle sprains and 59% having significant disability and residual symptoms causing impairment of their athletic performance. Ankle sprains were seen to occur in a myriad of sports ranging from basketball, jogging, fencing, racquetball, and almost any sport that involves running and jumping and at every level from recreational to the national team. It is not uncommon for athletes to be out for 2-3 weeks from a minor sprain and 6-10 weeks for a major sprain, and at least one study noted that an estimated 30% of all individuals who suffer an initial ankle sprain will develop chronic ankle instability.

Fear from future injury can also alter an athlete's professional career as evidenced by the careers of a number of notable NBA players. Over the course of many seasons, certain NBA players have sprained an ankle multiple times during NBA games and have undergone multiple ankle surgeries. At the professional level, the extensive history of injury makes it even more difficult for the athlete to obtain a long-term contract and may lead to premature retirement. Similar problems have arisen for star athletes in professional tennis competitions. Though often seen as trivial and short-term, ankle injuries may have extensive effects that plague an athlete throughout their entire career.

In addition, it has been noted that the plantar heel pain is the cause of over 1 million visits to physicians per year. Plantar fasciitis has been found to be the cause of 80% of all heel pain and can even lead to surgery in serious cases. Studies have also shown that obesity and reduced ankle dorsiflexion are signifying risk factors of the condition.

The athletic sock of the present invention incorporates reinforcing elastic material at specific locations around the feet, toes, heels and ankles that molds the athlete's foot into a dorsiflexed position that has been shown to simultaneously increases lateral foot speed and vertical jump height. Several studies have found that an athlete's squat jump, countermovement jump, continuous jump, speed at takeoff, and maximal and average power were all significantly higher when dorsiflexion was utilized. The problem is that dorsiflexion is not a natural foot position and it is strenuous for the athlete to dorsiflex, so it is difficult to maintain that position and remember to dorsiflex during competitions. As such, a device that can help maintain dorsiflexion, such as the one described here, would reduce the strain needed to dorsiflex and be of tremendous help in maintaining dorsiflexion whenever the athlete jumps or runs.

The elastic material also allows for a larger range of motion than an ankle brace while providing ankle protection. This reinforcing elastic material is emphasized along the ankle and the heel to provide support and along the foot to the pinky toe to dorsiflex that toe to help prevent lateral-extension ankle sprains, which account for approximately 80-90% of all ankle sprains. Lateral-extension ankle sprains typically occur when the pinky toe becomes immobilized on the ground, and the ankle is pushed to the outside, causing a forceful inward twisting of the bottom of the foot. Therefore, by raising the pinky toe in a dorsiflexed position, it helps prevent the pinky toe from being immobilized on the ground and to result in a lateral-extension ankle sprain. This implementation paired with additional support from elastic material that models an ankle brace around the ankle and heel will create a stable and secure sock.

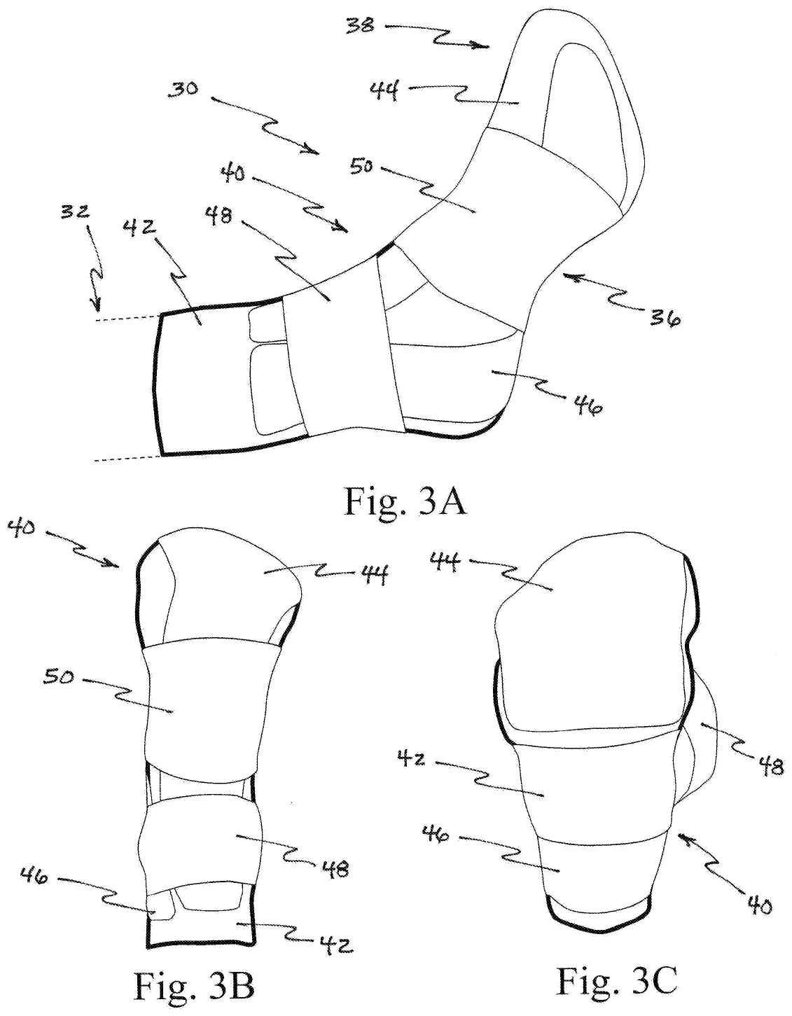

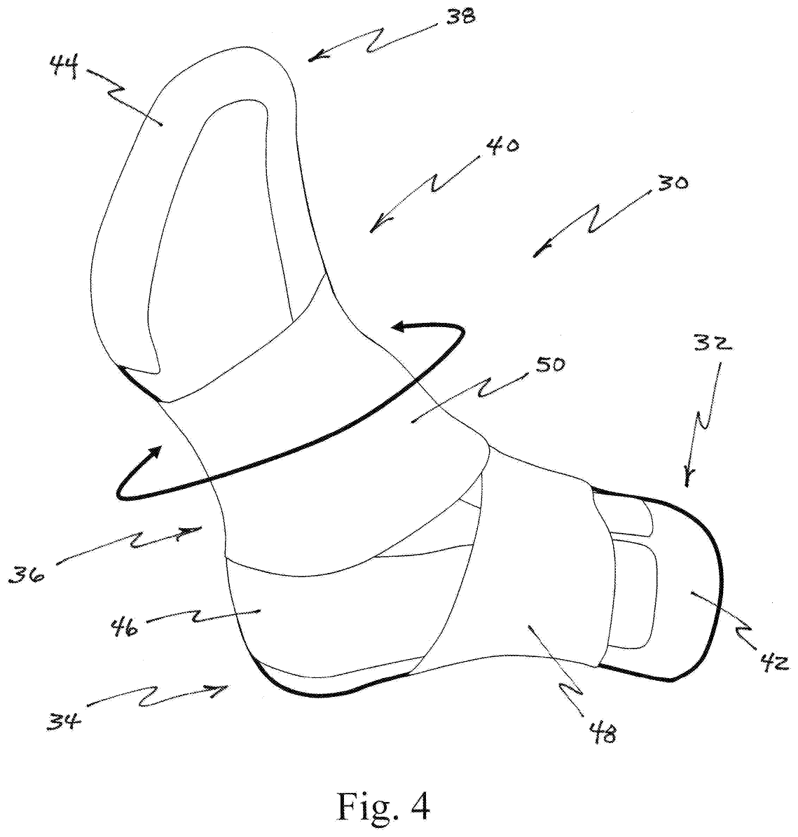

Furthermore, it turns out that dorsiflexion can reduce the likelihood of aggravating or bring relief to plantar fasciitis and foot and/or leg cramps. FIGS. 3A-3C & FIG. 4 show implementations of additional elastic material in one prototype that could be added as an option around the arch of the foot to provide additional support for the foot and help minimize pain from plantar fasciitis.

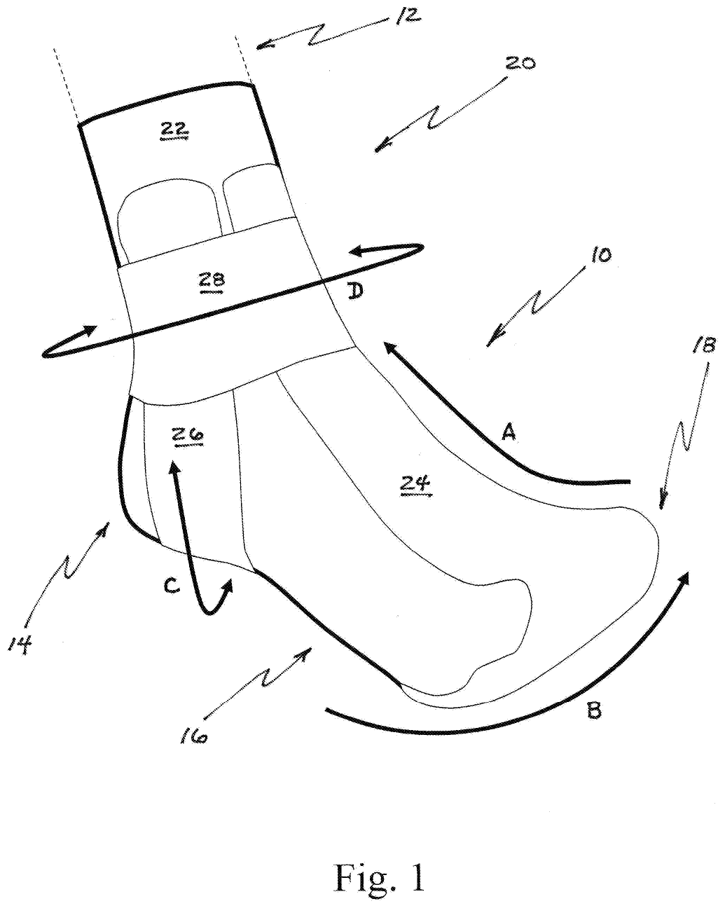

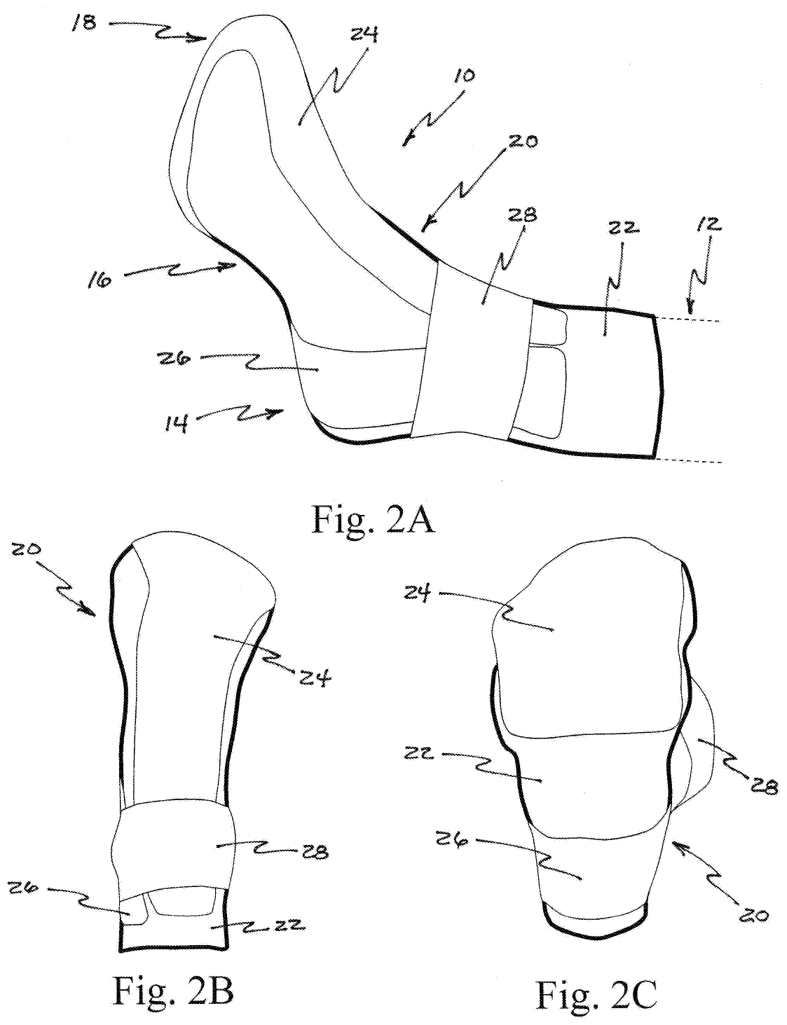

The first embodiment of the present invention as shown in FIG. 1 & FIGS. 2A-2C, utilizes kinesiology tape (of the type sold under the brand KT Tape) on basketball socks. It is understood that the areas of elastic shown in the appended drawings represent elastic material that has been adhered to the outer surface of an athletic sock. This representation is for clarity and it will be understood that implementation of increased elasticity areas on the sock of the present invention may take the form of elastics sections adhered to the sock (as shown) or as areas of increased elasticity incorporated into the material of the sock construction. For purposes of this detailed description of the embodiments, the areas of increased elasticity will be represented simply as adhered sections of kinesiology tape as was used in the prototype construction of the present invention.

Numerous tape placements and tensions have been tested, and an effective design has been developed as shown. FIG. 1 shows the first preferred embodiment where a foot 10 is positioned within the performance enhancing sock 20 of the present invention. As with ordinary athletic socks, sock 20 extends over the toe area 18 of foot 10, around the ball of the foot and over the instep 16 of the foot, and the heel 14 of the foot, up over the ankle area to a terminal top of the sock 10 positioned a distance up the leg 12 of the wearer.

The prototype of the first preferred embodiment of the present invention incorporated three seven-inch pieces of KT Tape that overlap side-by-side (elastic section 24 in FIG. 1) and pull from the balls of the feet to an area ranging from the instep of the foot to above the ankle to provide tension for dorsiflexion of the foot up to approximately 45 degrees (tension arrows A & B in FIG. 1). One seven-inch piece of KT Tape (elastic section 26 in FIG. 1) is placed under the foot and up both heels to provide ankle stability (tension arrow C in FIG. 1). Three eight-inch pieces of KT Tape (elastic section 28 in FIG. 1) placed directly on top of each other at low tension are placed around the ankle (tension arrow D in FIG. 1) to provide better support while allowing the sock to be easily worn and taken off. In some respects, this design models the formation of typical ankle brace for greater support and protection while maintaining a tensile strength that does not inhibit the foot's range of motion.

The design of the first preferred embodiment was tested in both vertical jump and lateral foot speed and initial results show an increase in 1-2 inches in vertical jump height and approximately a 3-5% increase in average sprint speed. While it could not be tested to see if it reduced the likelihood of ankle injuries since no ankle injuries were suffered during the initial testing phase, the sock did provide a more confident, secure and stable feeling around the ankle. Subjects suffering from plantar fasciitis describe some relief via these socks with and without the plantar fasciitis option (see FIGS. 3A-3C & FIG. 4). Finally, it could not be tested to see if it reduced the likelihood of foot and lower leg cramps since no cramps were suffered during the initial testing phase. While the sock may initially feel awkward since it molds the foot, once shoes are put on and the subject begins to run, it does not feel uncomfortable or cumbersome.

The preferred embodiment of the sock will preferably incorporate spandex in place of the KT Tape on the sock, however, other embodiments may use other elastic materials that will enact tension on the foot at the specified locations. Appropriate stretch materials include the following (some of which overlap in their definition and/or are trade names for certain products): Spandex and blends of spandex; Lycra and blends of Lycra; polyester; elastane; slinky knits; stretch satin; stretch velvet; Nylon and blends with Nylon; Rayon knit or blends with Spandex; bamboo Rayon; acetate/Spandex; modal/Spandex; tencel/Spandex; linen/Spandex; cotton poplin stretch; faux leather/vinyl fabric with stretch; Powernet; rubber/Latex; Neoprene rubber; and others. These elastic materials will serve a similar purpose to the KT Tape in causing dorsiflexion of the foot.

The preferred embodiment of the other parts 22 of the sock will be padded with additional cushioning and be made from a blend of fabrics to provide enhanced comfort and moisture-wicking properties. This part of the sock will be similar to pre-existing athletic socks.

FIG. 4 shows the second preferred embodiment where a foot 30 is positioned within the performance enhancing sock 40 of the present invention. As with ordinary athletic socks, sock 40 extends over the toe area 38 of foot 30, around the ball of the foot and over the instep 36 of the foot, and the heel 34 of the foot, up over the ankle area to a terminal top of the sock 30 positioned a distance up the leg 32 of the wearer.

The prototype of the second preferred embodiment of the present invention also incorporated three seven-inch pieces of KT Tape that overlap side-by-side (elastic section 44 in FIG. 4) and pull from the balls of the feet to an area ranging from the instep of the foot to above the ankle to provide tension for dorsiflexion of the foot up to approximately 45 degrees (similar to tension arrows A & B in FIG. 1). One seven-inch piece of KT Tape (elastic section 46 in FIG. 4) is placed under the foot and up both heels to provide ankle stability (similar to tension arrow C in FIG. 1). Three eight-inch pieces of KT Tape (elastic section 48 in FIG. 4) placed directly on top of each other at low tension are placed around the ankle (similar to tension arrow D in FIG. 1) to provide better support while allowing the sock to be easily worn and taken off. In some respects, this design models the formation of typical ankle brace for greater support and protection while maintaining a tensile strength that does not inhibit the foot's range of motion.

The further wrap of KT Tape (elastic section 50 in FIG. 4) provides the optional additional support associated with plantar fasciitis as described above.

Although the present invention has been described in terms of the foregoing preferred embodiments, this description has been provided by way of explanation only and is not intended to be construed as a limitation of the invention. Those skilled in the art will recognize modifications in the present invention that might accommodate specific sock structures and applications. Such modifications as to structure, method of manufacture, and even the specific arrangement of elastic components, where such modifications are coincidental to the application being addressed, do not necessarily depart from the spirit and scope of the invention.

I claim:

1. An athletic sock to help increase vertical jump height and lateral foot speed, reduce the likelihood of lateral-extension ankle sprains and foot and/or leg cramping, and bring relief to or reduce the likelihood of aggravating plantar fasciitis, comprising a sock body which includes a toe section, heel section, arch section and ankle section,
wherein the toe section comprises of elastic materials from the balls of the foot to an area around the instep of the foot, including the pinky toe;
wherein the heel section comprises of elastic materials under the heel in a U-shape;
wherein the arch section comprises of elastic materials around the arch of the foot;
wherein the leg section comprises of elastic materials around the ankle;
wherein the elastic materials in the toe section provides strong tension from the balls of the foot to an area around the instep of the foot to cause dorsiflexion of the toes of up to 45 degrees;
wherein the elastic materials in the heel section provides strong tension under the heel in a U shape for ankle stability and support;
wherein the elastic materials in the arch section provide strong tension around the arch of the foot for arch support; and
wherein the elastic materials of the leg section provide low tension around the ankle for ankle stability and support and does not prevent easy wearing of the sock or create discomfort or loss of ankle mobility;
such that said low tension is lower tension than said strong tension.

2. The sock according to the claim 1 wherein the tension among the toe, heel, arch and ankle sections are heterogeneous.

3. The sock according to claim 1 comprising a resilient elastic material and a natural or synthetic fiber.

4. The sock according claim 1 comprising the natural or synthetic fiber is selected from: cotton, wool, silk, microfiber, polyamide, or combinations thereof.

5. The sock according to claim 1 wherein the resilient elastic material is selected from: natural latex, a polyurethane-polyurea copolymer, spandex, elastane, nylon, neoprene, Lycra, polyester, or combinations thereof.
